(19) United States
(12) Patent Application Publication
(10) Pub. No.: US 2006/0002985 A1
(43) Pub. Date: Jan. 5, 2006

(54) Compositions and methods for decreasing age-related deterioration in mental activities in companion animals

(76) Inventors: Steven C. Zicker, Lawrence, KS (US); Larry H. Hayward, Topeka, KS (US); Dennis E. Jewell, Lawrence, KS (US)

Correspondence Address: HARNESS, DICKEY, & PIERCE, P.L.C, 7700 BONHOMME, STE 400, ST. LOUIS, MO, 63105, US

(21) Appl. No.: 10/884,732

(22) Filed: Jul. 2, 2004

(51) Int. Cl. A61K 31/198; A23K 1/165

(52) U.S. Cl. 424/442; 514/562

(57) ABSTRACT

Feline diet compositions comprising a sulfur-containing antioxidant are disclosed. The composition improves at least one behavioral attribute indicative of preventing or decreasing age-related deterioration of the mental capacity. Also disclosed are methods of preventing such deterioration by feeding the diet composition to a feline.

### CROSS REFERENCE TO RELATED APPLICATION

This application claims the benefit of U.S. application Ser. No. 10/613,604, filed on Jul. 3, 2003, which was converted on Jul. 1, 2004 into a provisional application (serial number unknown), having the same filing date of Jul. 3, 2003, and which is hereby incorporated in its entirety by reference.

### FIELD

The present invention relates to companion-animal diet compositions and, more particularly, to companion-animal diet compositions and methods for decreasing the deterioration in mental activities such as that associated with aging.

### BACKGROUND

Companion animals such as dogs and cats can suffer an age-related decline in mental activities associated with thinking, learning and memory (Houpt, K. A., Cognitive Dysfunction in Geriatric Cats, August J. R. ed, Consultations in Feline Internal Medicine 4^{th} ed., W.B. Saunders, Philadelphia, Pa. 2001, pp. 583-591). Additionally, behavioral changes and physical changes can be manifested in aging cats in association with the changes in mental capacity. Various possible causes thought to be responsible for this lessening of capacity have been advanced, however effective remedial approaches are not currently available.

### SUMMARY

Accordingly, the inventors herein have succeeded in discovering that the presence of one or more sulfur-containing antioxidants, in particular, sulfur-containing amino acids in the diet of a cat inhibits, i.e. prevents or reverses deterioration of mental capacity of the cat upon aging. Feeding a cat a diet composition that includes one or more the sulfur-containing other antioxidants, can bring about significantly increased physical activity of the cat.

Thus, in various embodiments the present invention can involve a method for decreasing deterioration of mental capacity in a feline. Decreasing deterioration of mental capacity includes either or both of preventing deterioration and reversing an existing state of deteriorated mental capacity. The method comprises feeding to the feline a diet composition comprising at least one sulfur-containing antioxidant and in particular, at least one sulfur-containing amino acid. The diet composition can further comprise at least one omega-3 fatty acid and an additional antioxidant selected from the group consisting of vitamin E, vitamin C, 1-carnitine and mixtures thereof. Feeding the diet prevents or reverses mental deterioration in the companion animal as a result of any one or more or all of the sulfur-containing antioxidants or sulfur-containing amino acids, omega-3 fatty acid and additional antioxidant being present in amounts that individually or together alter at least one behavioral attribute in a manner indicative of decreased deterioration of mental capacity.

The present invention, in various embodiments, can also include a feline diet composition. The composition can comprise at least one sulfur-containing antioxidant and, in particular, at least one sulfur-containing amino acid. The diet composition can further include at least one omega-3 fatty acid and at least two additional antioxidants selected from the group consisting of vitamin E, vitamin C, and 1-carnitine. The diet prevents or reverses mental deterioration in the companion animal as a result of any one or more or all of the sulfur-containing antioxidants or sulfur-containing amino acids, omega-3 fatty acid and additional antioxidant being present in amounts that individually or together alter at least one behavioral attribute in a manner indicative of decreased deterioration of mental capacity.

In various embodiments, the sulfur-containing amino acids or antioxidants in the methods and compositions of the present invention can include one or more sulfur-containing amino acid selected from the group consisting of cysteine, methionine, taurine, glutathione, s-adenosyl methionine, n-acetyl cysteine, cystathionine, cysteic acid, cysteine sulfinic acid, cystine, methionine sulfone, methionine sulfoxide, betaine, methyl hydroxy analog of methionine and mixtures thereof or a methyl ester of said sulfur-containing amino acids such as methionine methyl ester. In particular, the sulfur-containing amino acid or antioxidant can be methionine at a concentration of, for example, from about 0.8 wt. % to about 2.0 wt; cysteine at a concentration of, for example, from about 0.2 wt. % to about 0.7 wt. % or a mixture of cysteine and methionine at a concentration of from about 1.0 wt. % to about 2.2 wt. %. In various, but not all embodiments, the sulfur-containing antioxidant can be a sulfur-containing antioxidant other than lipoic acid.

In various embodiments, the altered behavioral attribute measured as an indication of decreased deterioration of mental capacity can be decreased wandering aimlessly, decreased inappropriate elimination, decreased excessive vocalization, increased initiating play, decreased sleeping, decreased purring, decreased demanding attention, decreased sitting in lap and/or increased physical activity.

In various embodiments, the omega-3 fatty acid can be docosahexaenoic acid, eicosapentaenoic acid, alpha-linolenic acid or combinations thereof at a concentration of at least about 50 ppm and the additional antioxidant can be vitamin E at a concentration of at least about 500 ppm, vitamin C at a concentration of at least about 50 ppm and 1-carnitine at a concentration of at least about 100 ppm (on a dry matter basis).

The companion cat diet meets ordinary nutritional requirements of the cat of at least one year of age

### DETAILED DESCRIPTION

The diet fed to an aging companion cat, can a standard normal diet such as that shown in Table 1 for feeding to an adult cat.

TABLE 1

| Component | Target |
|---|---|
| Protein (% of dry matter) | ≧30 |
| Fat (% of dry matter) | ≧10 |
| Phosphorous (% of dry matter) | ≧0.5 |
| Sodium (% of dry matter) | ≧0.2 |

Similar diets can be fed to adult cats of greater than about one year of age and older cats of about 7 years of age or greater.

Adding significant quantities of a sulfur-containing antioxidant, in particular, a sulfur-containing amino acid to the companion cat diet can bring about significant and demonstrative changes in the behavior, as specifically shown by increased overall physical activity in an aged cat or by preventing such occurrence of decreased physical activity upon feeding the diet to an adult cat of greater than one year of age. The term, aged is intended to mean, in general, a cat of at least seven years.

The loss of mental capacity with age can be manifested in numerous ways. In a cat, for example, it can be manifested as disorientation, house soiling, altered sleep-wake patterns, decreased interaction with family members and pets, excessive vocalization and diminished physical activity.

By administering the diet of the present invention, an aging cat's physical activity can be enhanced. Although the present diet composition can be used to prevent or reverse age-related deterioration in mental activities, deterioration in mental activities due to causes other than aging such as deterioration due to neurodegenerative diseases can also be prevented or reversed.

A dietary antioxidant, or precursor thereof, can be defined as “a substance in foods that significantly decreases the adverse effects of reactive species, such as reactive oxygen and nitrogen species, on normal physiological function in humans”. (Dietary Reference Intakes of Vitamin C, Vitamin E, Selenium, and Carotenoids, Food and Nutrition Board Institute of Medicine, National Academy Press, Washington, D.C., April, 2000, p. 42, said reference being incorporated in its entirety by reference).

Numerous antioxidants can be found in nature and many of such antioxidants are sulfur-containing antioxidants. For example, while not intending to be bound by any theoretical mechanism of action, the sulfur-containing amino acid, methionine, is believed to possess free-radical scavenging activity by virtue of its containing a sulfur which is oxidizable, as well as its having chelating ability. Methionine can also serve as precursor of other antioxidant compounds such as, for example, cysteine. As another example, the sulfur-containing amino acid, cysteine, also contains an oxidizable sulfur and this amino acid can serve as a precursor of the antioxidant glutathione. In a further example of a sulfur-containing antioxidant, the sulfonic amino acid, taurine, is believed to act as an antioxidant by reacting with excess hypochlorite produced in the process of phagocytosis to form N-chlorotaurine. Specific, non-limiting examples of sulfur containing antioxidants include sulfur-containing amino acids such as cysteine, methionine taurine, glutathione, s-adenosyl methionine, n-acetyl cysteine, cystathionine, cysteic acid, cysteine sulfinic acid, cystine, methionine sulfone methionine sulfoxide, betaine, methyl hydroxy analog of methionine, sulfur containing amino acids in addition to those listed above, methyl esters of amino acids such as methionine methyl ester, and the like including other sulfur-containing substance exhibiting the properties described above.

The sulfur-containing antioxidants or sulfur-containing amino acids of the present invention can be naturally occurring or synthetic substances. In various embodiments, the sulfur-containing antioxidants include antioxidants other than lipoic acid.

Other antioxidants can also be present in the diet compositions of the present invention. Such antioxidants include vitamin E, vitamin C and 1-carnitine. Additionally, omega-3 fatty acids can also be present. Vitamin E can be administered as a tocopherol or a mixture of tocopherols and various derivatives thereof such as esters like vitamin E acetate, succinate, palmitate, and the like. The alpha form is preferable but beta, gamma and delta forms can be included. The d form is preferable but racemic mixtures are acceptable. The forms and derivatives will function in a vitamin E like activity after ingestion by the pet. Vitamin C can be administered in this diet as ascorbic acid and its various derivatives thereof such as calcium phosphate salts, cholesteryl salt, 2-monophosphate, and the like which will function in a vitamin C like activity after ingesting by the pet. The above vitamins can be in any form such as liquid, semisolid, solid and heat stable form. L-carnitine can be administered in the diet and various derivatives of carnitine such as the salts such as the hydrochloride, fumarate and succinates, as well as acetylated carnitine, and the like can be used. Omega-3 fatty acids are found in natural sources such as fish oil or vegetable matter. Omega-3 fatty acids found in nature include eicosapentaenoic acid (EPA), docosahexaenoic acid (DHA) and alpha-linolenic acid (ALA).

The diet compositions of the present invention can be in the form of wet cat foods, semi-moist cat foods, dry cat foods and cat treats. Wet cat food generally has a moisture content greater than about 65%. Semi-moist cat food typically has a moisture content between about 20% and about 65% and may include humectants, potassium sorbate, and other ingredients to prevent microbial growth (bacteria and mold). Dry cat food (kibble) generally has a moisture content below about 10% and its processing typically includes extruding, drying and/or baking in heat. Cat treats typically may be semi-moist, chewable treats; dry treats in any number of forms; chewable bones or baked, extruded or stamped treats; confection treats; or other kinds of treats as is known to one skilled in the art.

The quantities administered in a nutritionally balanced diet, all as wt % (dry matter basis) of the diet, are calculated as the active material, per se, that is measured as free material. The maximum amounts employed should not bring about toxicity.

Methionine can be present in the diet compositions of the present invention at a concentration of at least about 0.8 wt. %, at least about 0.9% wt. %, at least about 1.0 wt. %, at least about 1.1 wt. % up to about 1.5% or greater. Cysteine can be present in the diet compositions of the present invention at concentrations of at least about 0.2 wt. %, at least about 0.3 wt. %, at least about 0.4 wt. %, at least about 0.5% up to about 0.7% or greater. The combination of methionine and cysteine can also be present at a total concentration of sulfur amino acids of at least about at least about 1.0 wt. %, at least about 1.2 wt. %, at least about 1.4 wt. %, at least about 1.5 wt. %, at least about 1.6 wt. %, up to about 2.2 wt. %.

Vitamin E can be present at a concentration of at least about 100 ppm, at least about 150 ppm, at least about 300 ppm, at least about 500 ppm on a weight basis up to a maximum non-toxic concentration. Maximum concentration of vitamin E can be up to about 1,000 ppm, up to about 1,500 ppm, up to about 2,000 ppm on a weight basis or greater.

Vitamin C can be present at a concentration of at least about 50 ppm, at least about 75 ppm, at least about 100 ppm, at least about 200 ppm up on a weight basis up to a maximum non-toxic concentration. Maximum concentration of vitamin C can be up to about 500 ppm, up to about 600 ppm on a weight basis or greater.

In various embodiments, the diet compositions of the present invention can also contain 1-carnitine at a concentration of at least about 100 ppm, at least about 200 ppm, or at least about 500 ppm on a weight basis up to a maximum non-toxic concentration. Maximum concentration of 1-carnitine can be up to about 4,000 ppm, up to about 5,000 ppm, up to about 6,000 on a weight basis or greater.

Omega-3 fatty acids can be present in the diet compositions at concentrations of at least about 0.05% wt. %, at least about 0.1 wt. %, at least about 0.2 wt. %, at least about 0.3 wt. % up to a maximum non-toxic concentration. Typically, one or more omega-3 fatty acids can be present in the diet at a concentration at a total concentration of omega-3 fatty acids of about 0.3 wt. %, about 0.4 wt. % or about 0.5 wt. % or greater.

Other materials can also be present if desired. These materials and their quantities are shown below:
Beta-carotene at about 1-15 ppm can be employed.
Selenium at about 0.1 up to about 5 ppm can be employed.
Lutein at least about 5 ppm can be employed.
Tocotrienols at least about 25 ppm can be employed.
Coenzyme Q10 at least about 25 ppm can be employed.
Soy isoflavones at least about 25 ppm can be used.
Ginkgo Biloba at least 50 ppm of extract or 1% of diet can be used.

### EXAMPLE

This study illustrates the effect of a diet containing increased amounts of methionine and cysteine along with increased amounts of vitamin E, vitamin C, 1-carnitine and the omega-3 fatty acids, docosahexaenoic acid, eicosapentaenoic acid, and alpha-linolenic acid on behavioral characteristics indicative of mental deterioration associated with aging.

Aged cats were enrolled into the trial at 3 different veterinary hospital sites. Older cats of approximately 10 years of age were screened for physical health, clinical bloodwork (to assess liver, thyroid, kidney, endocrine and other health indices), and urinary tract health. Cats were required to have no overt health problems based on the physical and other evaluations. Owners were then asked to assess the behavior of their cat compared to when it was 8 years of age in 22 behavioral attributes.

Forty-six (46) cats fulfilled the health requirements and were enrolled into the study. Cats were assigned randomly to either a test or control food and re-evaluated by questionnaire after 30 days of feeding the assigned food. Both owners and the veterinarian overseeing the trial were masked to the identity of the food assigned. Food compositions were as shown below in Table 2.

TABLE 2
Concentration of Key Nutrients in Feline Test and Control
Formulas in wt % on a dry matter basis

| Analyte | Test | Control |
|---|---|---|
| Protein | 33.5 | 32.8 |
| Fat | 18.1 | 19.8 |
| Calcium | 1.02 | 0.909 |
| Phosphorous | 0.66 | 0.65 |
| Magnesium | 0.069 | 0.067 |
| Taurine | 0.2161 | 0.1946 |
| Vitamin E (IU/kg) | 1178 | 114 |
| Vitamin C (ppm) | 175 | <10 |
| Camitine (ppm) | 562 | 15 |
| Docosahexaenoic acid | 0.123 | 0.026 |
| Eicosapentaenoic acid | 0.11 | 0.024 |
| alpha-linolenic | 0.279 | 0.119 |
| Methionine | 1.29 | 0.932 |
| Cysteine | 0.61 | 0.475 |
| Met+Cys | 1.902 | 1.407 |

Baseline and 30 day data from 46 enrolled cats was assessed by statistical analysis for between group and within group differences. Baseline data showed no significance between group differences for all questions asked at the start of the study. However, significant differences were present in 9 of the 22 current behavioral assessments asked of owners in comparison to when their cat was 8 years of age (Table 3).

TABLE 3
Baseline Data Compared to Cats 8 Years of Age

| Question | Significant Change |
|---|---|
| Wandering aimlessly | More |
| Inappropriate elimination | More |
| Excess vocalization | More |
| Initiate play | Less |
| Sleeping | More |
| Purring | More |
| Demand attention | More |
| Sit in lap | More |
| Active | Less |

Upon completion of the 30 day feeding time point of the study, analysis of the results revealed a significant increase in one behavioral assessment, increased activity, in the group receiving test diet compared to the value in the group receiving control diet. This result, coupled with the baseline data, showed that the owners noticed an increased activity similar to that of a younger animal within 30 days of dietary change. All references cited in this specification are hereby incorporated by reference in their entirety. The discussion of the references herein is intended merely to summarize the assertions made by their authors and no admission is made that any reference constitutes prior art relevant to patentability. Applicant reserves the right to challenge the accuracy and pertinency of the cited references.

What is claimed is:

1. A method for decreasing deterioration of mental capacity in a feline, the method comprising feeding to the feline a diet composition comprising at least one sulfur-containing antioxidant, at least one omega-3 fatty acid and an additional antioxidant selected from the group consisting of vitamin E, vitamin C, 1-carnitine and mixtures thereof, wherein the sulfur-containing antioxidant, omega-3 fatty acid and additional antioxidant are present in the diet composition in amounts that together alter at least one behavioral attribute in a manner indicative of decreased deterioration of mental capacity.

2. A method according to claim 1, wherein the at least one sulfur-containing antioxidant is at least one sulfur-containing antioxidant other than lipoic acid.

3. A method according to claim 1, wherein the sulfur-containing antioxidant is one or more sulfur-containing amino acid selected from the group consisting of cysteine, methionine, taurine, glutathione, s-adenosyl methionine, n-acetyl cysteine, cystathionine, cysteic acid, cysteine sulfinic acid, cystine, methionine sulfone, methionine sulfoxide, betaine, methyl hydroxy analog of methionine and mixtures thereof or a methyl ester of one or more said sulfur-containing amino acids such as methionine methyl ester.

4. A method according to claim 3, wherein the at least one sulfur-containing antioxidant is methionine, cysteine or a mixture of cysteine and methionine.

5. A method according to claim 4, wherein the methionine is present at a concentration of from about 0.8 wt. % to about 1.5 wt. %.

6. A method according to claim 4, wherein the cysteine is present in an amount of from about 0.2 wt. % to about 0.7 wt. %.

7. A method according to claim 4, wherein the mixture of cysteine and methionine is present in a total amount of from about 1.0 wt. % to about 2.2 wt. %.

8. A method according to claim 1, wherein the at least one altered behavioral attribute is selected from the group consisting of decreased wandering aimlessly, decreased inappropriate elimination, decreased excessive vocalization, increased initiating play, decreased sleeping, decreased purring, decreased demanding attention, decreased sitting in lap and increased physical activity.

9. A method according to claim 8, wherein the at least one altered behavioral attribute is increased physical activity.

10. A method according to claim 1, wherein the additional antioxidant comprises vitamin E at a concentration of at least about 500 ppm, vitamin C at a concentration of at least about 50 ppm and 1-carnitine at a concentration of at least about 100 ppm.

11. A method according to claim 1, wherein the at least one omega-3 fatty acid is selected from the group consisting of docosahexaenoic acid, eicosapentaenoic acid, alpha-linolenic acid and combinations thereof.

12. A method according to claim 1, wherein the at omega-3 fatty acid is present in the diet composition at a concentration of at least about 0.05 ppm.

13. A feline diet composition comprising at least one sulfur-containing antioxidant, at least one omega-3 fatty acid and at least two additional antioxidants selected from the group consisting of vitamin E, vitamin C, and 1-carnitine, wherein the sulfur-containing antioxidant, omega-3 fatty acid and additional antioxidant are present in the diet composition in amounts that together alter at least one behavioral attribute in a manner indicative of decreased deterioration of mental capacity.

14. A composition according to claim 13, wherein the at least one sulfur-containing antioxidant is at least one sulfur-containing antioxidant other than lipoic acid.

15. A composition according to claim 13, wherein the sulfur-containing antioxidant is one or more sulfur-containing amino acid selected from the group consisting of cysteine, methionine, taurine, glutathione, s-adenosyl methionine, n-acetyl cysteine, cystathionine, cysteic acid, cysteine sulfinic acid, cystine, methionine sulfone, methionine sulfoxide, betaine, methyl hydroxy analog of methionine and mixtures thereof or a methyl ester of one or more said sulfur-containing amino acids such as methionine methyl ester.

16. A composition according to claim 15, wherein the at least one sulfur-containing antioxidant is methionine, cysteine or a mixture of cysteine and methionine.

17. A composition according to claim 16, wherein the methionine is present at a concentration of from about 0.8 wt. % to about 2.0 wt. %.

18. A composition according to claim 16, wherein the cysteine is present in an amount of from about 0.2 wt. % to about 0.7 wt. %.

19. A composition according to claim 16, wherein the mixture of cysteine and methionine is present in a total amount of from about 1.0 wt. % to about 2.2 wt. %.

20. A composition according to claim 13, wherein the at least one altered behavioral attribute is selected from the group consisting of decreased wandering aimlessly, decreased inappropriate elimination, decreased excessive vocalization, increased initiating play, decreased sleeping, decreased purring, decreased demanding attention, decreased sitting in lap and increased physical activity.

21. A composition according to claim 20, wherein the at least one altered behavioral attribute is increased physical activity.

22. A composition according to claim 13, wherein the at least two additional antioxidant comprises vitamin E at a concentration of at least about 500 ppm, vitamin C at a concentration of at least about 50 ppm and 1-carnitine at a concentration of at least about 100 ppm.

23. A composition according to claim 13, wherein the at least one omega-3 fatty acid is selected from the group consisting of docosahexaenoic acid, eicosapentaenoic acid, alpha-linolenic acid and combinations thereof.

24. A composition according to claim 13, wherein the omega-3 fatty acid is present in the diet composition at a concentration of at least about 0.05 ppm.
